(19) United States
(12) Patent Application Publication
(10) Pub. No.: US 2026/0041324 A1
(43) Pub. Date: Feb. 12, 2026

(54) Assessment Method of Vascular Function in Dialysis Patient and System Thereof

(71) Applicants: Asia University, Taichung City (TW); China Medical University, Kaohsiung City (TW)

(72) Inventors: Chiu-Chin Huang, Taipei City (TW); Charles C. N. Wang, Kaohsiung City (TW); Ping Chin Lai, Taichung City (TW); Huey-Liang Kuo, Taichung City (TW); Kai-Ling Yang, Taichung City (TW)

(21) Appl. No.: 19/295,601

(22) Filed: Aug. 9, 2025

(30) Foreign Application Priority Data
Aug. 9, 2024 (TW) 113130046

(51) Int. Cl. A61B 5/02; A61B 5/00; A61B 5/021; A61M 1/16

(52) U.S. Cl. CPC A61B 5/02007; A61B 5/021; A61B 5/7264; A61B 5/746; A61M 1/16; A61B 2562/0247; A61M 2202/0021; A61M 2202/0413; A61M 2205/18; A61M 2205/3303; A61M 2205/3327; A61M 2205/3331; A61M 2205/3592; A61M 2205/502; A61M 2210/12; A61M 2230/30

(57) ABSTRACT

An assessment method for vascular function of a dialysis patient and system thereof are provided. The assessment method measures a venous end vascular pressure of the dialysis patient, and if the venous end vascular pressure of the dialysis patient exceeds a preset pressure variation range within a preset time, blood vessels of the dialysis patient are evaluated for a presence of at least one abnormality. The assessment system includes a hemodialysis device and an output device.

### CROSS-REFERENCE TO RELATED APPLICATION

This application claims priority to Taiwan Patent Application No. 113130046, filed on Aug. 9, 2024, in the Taiwan Intellectual Property Office, the disclosure of which is incorporated herein in its entirety by reference.

### BACKGROUND OF THE INVENTION

### 1. Field of the Invention

The present disclosure relates to an assessment method for vascular function and a system thereof, and more particularly to an assessment method for vascular function of a dialysis patient and a system thereof.

### 2. Description of the Related Art

Patients with chronic kidney disease often require long-term hemodialysis treatment to sustain life. During dialysis, the needles of the hemodialysis device are inserted into the patient's dedicated vascular access, commonly referred to as a fistula or arteriovenous fistula. However, prolonged dialysis may lead to various complications in the arteriovenous fistula, such as vascular stenosis, occlusion, or sclerosis. These vascular problems not only affect the effectiveness of dialysis but may also pose a life-threatening risk to the patient. Therefore, timely detection and assessment of functional abnormalities in the arteriovenous fistula of dialysis patients are critically important.

Existing methods for evaluating the vascular function of dialysis patients, such as ultrasound examinations and angiography, often require additional examination time and equipment, making it difficult to perform real-time assessments during dialysis. Moreover, when blood flow is continuous, vascular abnormalities may not be easily detected because blood pressure can remain relatively stable even in the presence of such issues.

Accordingly, it is essential to develop a method that enables simple, rapid, and accurate evaluation of vascular function during the dialysis process.

### SUMMARY OF THE INVENTION

In view of the foregoing issues, the inventors of the present disclosure have contemplated and developed an assessment method for vascular function of a dialysis patient and system thereof, with a view to addressing the problem in the prior art that vascular abnormalities in dialysis patients cannot be detected in a timely manner.

An object of the present disclosure is to provide an assessment method for vascular function of a dialysis patient and system thereof, which enables rapid and effective identification of whether vascular abnormalities are present in dialysis patients during the dialysis session.

According to one aspect of the present disclosure, an assessment method for vascular function of a dialysis patient is provided, and the assessment method includes the following steps:
Step S1: measuring a venous end vascular pressure of the dialysis patient by a hemodialysis device, and displaying the venous end vascular pressure on an output device.
Step S2: detecting a pressure change of the venous end vascular pressure of the dialysis patient within a preset time.
Step S3: in response to the pressure change of the venous end vascular pressure of the dialysis patient exceeding a preset pressure change range, generating a warning signal.
Step S4: transmitting the warning signal to the output device and/or a communication device.

The venous end vascular pressure of the dialysis patient in the step S2 and the step S3 refers to the venous end vascular pressure measured when blood is continuously returned through a venous end after a completion of a dialysis session and blood withdrawal from an arterial end is stopped.

In the step S2, the venous end vascular pressure of the dialysis patient is detected by a blood pressure monitoring sensor installed in the hemodialysis device, and the preset time ranges from 5 to 10 minutes.

In the step S3, the preset pressure change range refers to an increase or decrease of at least 50% relative to an average vascular pressure value of venous end vessels of the dialysis patient; and when the venous end vascular pressure exceeds the preset pressure change range, the blood pressure monitoring sensor generates the warning signal.

Furthermore, when the warning signal is generated, blood vessels of the dialysis patient are evaluated for a presence of at least one abnormality, and the abnormal condition is selected from vascular stenosis, vascular occlusion and vascular sclerosis.

Finally, in step S4, by transmitting the warning signal to the output device and/or the communication device, physicians and medical personnel can further examine the blood vessels of the dialysis patient in response to the warning signal to determine the type of vascular abnormality present, and subsequently perform corresponding treatment to address the vascular abnormality. Alternatively, when the dialysis patient is undergoing home hemodialysis and the warning signal is generated, the communication device can receive the warning signal and report it to the hospital, thereby facilitating the arrangement of an examination to determine the type of vascular abnormality present in the dialysis patient's blood vessels and to perform corresponding treatment to resolve the vascular abnormality.

According to another object of the present disclosure, an assessment system for vascular function of a dialysis patient is provided. The assessment system includes a hemodialysis device and an output device; the hemodialysis device at least includes a dialysis apparatus, a blood pressure monitoring sensor, a transmission terminal, and a wireless transmission device, the output device is connected to the hemodialysis device via the transmission terminal, the blood pressure monitoring sensor is electrically connected to the dialysis apparatus, the transmission terminal, and the wireless transmission device.

The dialysis apparatus is configured to perform a dialysis session on the dialysis patient, and during the dialysis session, a blood pressure of the dialysis patient is monitored using the blood pressure monitoring sensor, and a blood pressure monitoring status of the dialysis patient is displayed on the output device via the transmission terminal; and after a completion of the dialysis session, the following steps are performed:
detecting a pressure change of a venous end vascular pressure of the dialysis patient within a preset time; in response to the pressure change of the venous end vascular pressure of the dialysis patient exceeding a preset pressure change range, generating a warning signal through the blood pressure monitoring sensor; and transmitting the warning signal to the output device and/or a communication device.

The venous end vascular pressure of the dialysis patient in the steps performed refers to the venous end vascular pressure measured when blood is continuously returned through a venous end after a completion of a dialysis session and blood withdrawal from an arterial end is stopped.

The preset time ranges from 5 to 10 minutes.

The preset pressure change range refers to an increase or decrease of at least 50% relative to an average vascular pressure value of venous end vessels of the dialysis patient.

The output device may include various display interfaces, such as computer screens, monitors, or handheld device displays, but is not limited thereto.

The communication device refers to a device with wired or wireless transmission capabilities, such as desktop computers, laptop computers, tablet computers, smartphones, and the like, but is not limited thereto.

When the warning signal is generated, it may be transmitted to the output device via the transmission terminal, or alternatively, it may be transmitted to the communication device via the wireless transmission device. For example, an application (APP) installed on the communication device may be used to receive the warning signal. This enables physicians and medical personnel to further examine the blood vessels of the dialysis patient in response to the warning signal, to determine the type of vascular abnormality present and subsequently perform appropriate treatment to address the vascular abnormality.

On the other hand, when the dialysis patient is undergoing home hemodialysis and the warning signal is generated, the communication device can receive the warning signal and report it to the hospital, thereby facilitating the arrangement of an examination to determine the type of vascular abnormality present in the dialysis patient's blood vessels and subsequently perform appropriate treatment to resolve the vascular abnormality.

The advantages of the present disclosure are as follows:
1. Simplicity and speed: The method can be performed during a routine dialysis session without requiring additional examination time or equipment.
2. High sensitivity: By observing vascular pressure changes within a short period after stopping arterial blood withdrawal, vascular abnormalities can be detected more sensitively.
3. Real-time capability: Vascular issues can be identified immediately at the end of dialysis, facilitating timely intervention and treatment.
4. Non-invasiveness: No additional invasive procedures are required, thereby reducing discomfort and risk for dialysis patients.
5. Cost-effectiveness: Expensive dedicated equipment is not needed; only the hemodialysis device is required, enabling widespread application in medical institutions of all levels and even in home hemodialysis settings.

The following detailed description, in conjunction with the accompanying drawings, will further illustrate specific embodiments to facilitate understanding of the objectives, technical content, features, and advantages of the present disclosure.

### BRIEF DESCRIPTION OF THE DRAWINGS

To make the technical features, content, advantages, and effects achievable by the present disclosure more apparent, the present disclosure will now be described in detail with reference to the accompanying drawings and through exemplary embodiments as follows:

FIG. 1 is a flowchart of an assessment method for vascular function of a dialysis patient according to the present disclosure;

FIG. 2 is a graph showing venous end vascular pressure variations of Patient A (Case 1) before undergoing percutaneous arteriovenous fistula angioplasty;

FIG. 3 is a graph showing venous end vascular pressure variations of Patient A (Case 1) after undergoing percutaneous arteriovenous fistula angioplasty;

FIG. 4 is a graph showing venous end vascular pressure variations of Patient B (Case 2) before undergoing percutaneous arteriovenous fistula angioplasty;

FIG. 5 is a graph showing venous end vascular pressure variations of Patient B (Case 2) after undergoing percutaneous arteriovenous fistula angioplasty;

FIG. 6 is a graph showing venous end vascular pressure variations of Patient C (Case 3);

FIG. 7 is a graph showing venous end vascular pressure variations of Patient D (Case 4); and

FIG. 8 is a schematic diagram of the system for evaluating vascular function in dialysis patients according to the present disclosure.

### DETAILED DESCRIPTION OF THE PREFERRED EMBODIMENTS

The following contents will be accompanied by drawings to illustrate the technical contents of the present disclosure through specific embodiments. People familiar with this technology can easily understand other advantages and effects of the present disclosure from the contents disclosed in this specification. The present disclosure may also be implemented or applied through other different specific embodiments. Various modifications and changes may be made to the details in this specification based on different viewpoints and applications without departing from the spirit of the present disclosure.

Unless otherwise defined, all terms (including technical and scientific terms) used in the present disclosure have the same meaning as commonly understood by one of ordinary skill in the art to which the present disclosure belongs. It will be further understood that those terms as defined in commonly used dictionaries should be interpreted as having definitions that are consistent with their meanings in the context of the relevant art and the present disclosure, and will not be interpreted as idealized or overly formal meanings unless expressly so defined herein.

All numbers herein are understood to be modified by the word “about”. As used herein, the term “about”is intended to encompass variations of ±10%.

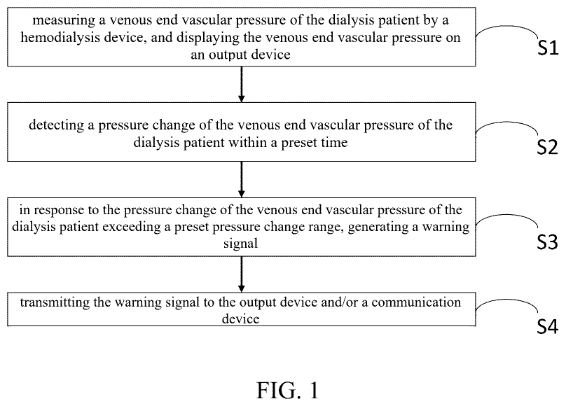

Referring to FIG. 1, FIG. 1 is a flowchart of an assessment method for vascular function of a dialysis patient according to the present disclosure. As shown in FIG. 1, an assessment method for vascular function of a dialysis patient includes the following steps:
Step S1: measuring a venous end vascular pressure of the dialysis patient by a hemodialysis device, and displaying the venous end vascular pressure on an output device.
Step S2: detecting a pressure change of the venous end vascular pressure of the dialysis patient within a preset time.
Step S3: in response to the pressure change of the venous end vascular pressure of the dialysis patient exceeding a preset pressure change range, generating a warning signal.
Step S4: transmitting the warning signal to the output device and/or a communication device.

The venous end vascular pressure of the dialysis patient in the step S2 and the step S3 refers to the venous end vascular pressure measured when blood is continuously returned through a venous end after a completion of a dialysis session and blood withdrawal from an arterial end is stopped.

In the step S2, the venous end vascular pressure of the dialysis patient is detected by a blood pressure monitoring sensor installed in the hemodialysis device, and the preset time ranges from about 5 to about 10 minutes; preferably, the preset time ranges from about 6 to about 8 minutes.

Furthermore, in the step S3, the preset pressure change range refers to an increase or decrease of at least 50% relative to an average vascular pressure value of venous end vessels of the dialysis patient; preferably, the preset pressure change range refers to an increase or decrease of at least 80% relative to an average vascular pressure value of venous end vessels of the dialysis patient; and when the venous end vascular pressure exceeds the preset pressure change range, the blood pressure monitoring sensor generates the warning signal.

Furthermore, when the warning signal is generated, blood vessels of the dialysis patient are evaluated for a presence of at least one abnormality, and the abnormal condition is selected from vascular stenosis, vascular occlusion and vascular sclerosis.

Finally, in step S4, by transmitting the warning signal to the output device and/or the communication device, physicians and medical personnel can further examine the blood vessels of the dialysis patient in response to the warning signal to determine the type of vascular abnormality present, and subsequently perform corresponding treatment to address the vascular abnormality. Alternatively, when the dialysis patient is undergoing home hemodialysis and the warning signal is generated, the communication device can receive the warning signal and report it to the hospital, thereby facilitating the arrangement of an examination to determine the type of vascular abnormality present in the dialysis patient's blood vessels and to perform corresponding treatment to resolve the vascular abnormality.

The following provides a description of specific operations performed on actual dialysis patients to demonstrate the concrete effects achievable by the assessment method for vascular function of the dialysis patient of the present disclosure.

### Experimental Example 1: Abnormal Increase in Venous end Vascular Pressure

Patient A (Case 1) first undergoes a dialysis treatment. After completion of the dialysis session, the hemodialysis device is controlled to stop withdrawing blood from the arterial end while continuing to return blood through the venous end, and the vascular pressure variations at the venous end of Patient A are monitored.

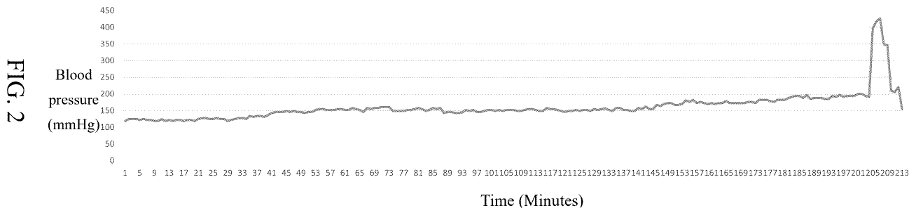

Referring to FIG. 2, FIG. 2 is a graph showing venous end vascular pressure variations of Patient A (Case 1) before undergoing percutaneous arteriovenous fistula angioplasty. As shown in FIG. 2, during the dialysis session, the blood pressure of Patient A remained stable. After completion of the dialysis session, the hemodialysis device is controlled to stop withdrawing blood from the arterial end while continuing to return blood through the venous end. Subsequently, between 204 and 209 minutes, a rapid increase in venous end vascular pressure is observed, indicating that Patient A's blood vessels may have an abnormal condition, warranting further investigation to determine the cause of the sudden rise in the venous end vascular pressure.

After identifying the cause of the vascular abnormality, percutaneous arteriovenous fistula angioplasty is performed on Patient A to address the issue. Thereafter, Patient A undergoes another dialysis treatment.

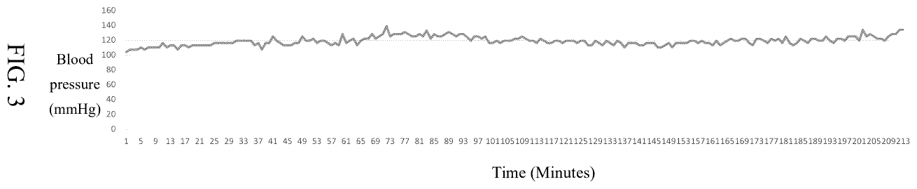

Referring to FIG. 3, FIG. 3 is a graph showing venous end vascular pressure variations of Patient A (Case 1) after undergoing percutaneous arteriovenous fistula angioplasty. As shown in FIG. 3, the venous end vascular pressure remained stable, indicating that the vascular abnormality is successfully resolved after the surgery.

### Experimental Example 2: Abnormal Decrease in Venous end Vascular Pressure

In the same manner as Experimental Example 1, Patient B (Case 2) first undergoes a dialysis treatment. After completion of the dialysis session, the hemodialysis device is controlled to stop withdrawing blood from the arterial end while continuing to return blood through the venous end, and the vascular pressure variations at the venous end of Patient B are monitored.

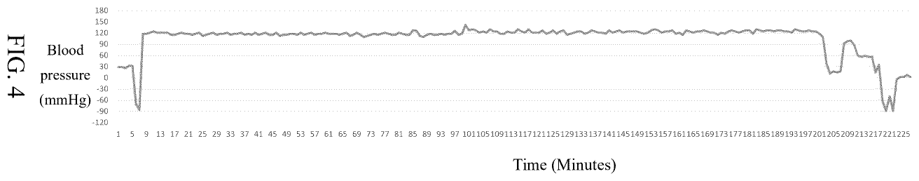

Referring to FIG. 4, FIG. 4 is a graph showing venous end vascular pressure variations of Patient B (Case 2) before undergoing percutaneous arteriovenous fistula angioplasty. As can be seen from FIG. 4, during the dialysis session, the blood pressure of Patient B remained stable. After completion of the dialysis session, the hemodialysis device is controlled to stop withdrawing blood from the arterial end while continuing to return blood through the venous end. Subsequently, two rapid drops in venous end vascular pressure are observed between 202-208 minutes and 218-224 minutes, indicating that Patient B's blood vessels may have an abnormal condition, requiring further investigation to determine the cause of the sudden decreases in venous end vascular pressure.

Similarly, after identifying the cause of the vascular abnormality, percutaneous arteriovenous fistula angioplasty is performed on Patient B to address the issue. Thereafter, Patient B undergoes another dialysis treatment.

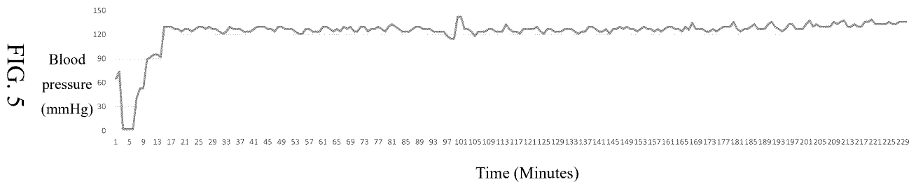

Referring to FIG. 5, FIG. 5 is a graph showing venous end vascular pressure variations of Patient B (Case 2) after undergoing percutaneous arteriovenous fistula angioplasty. As shown in FIG. 5, the venous end vascular pressure remained stable, indicating that the vascular abnormality is successfully resolved after the surgery.

In addition, in FIGS. 4 and 5, the decrease in venous end vascular pressure observed during approximately 1 to 10 minutes is caused by short-term blood pressure fluctuations at the beginning of the dialysis session, when blood withdrawal from the arterial end and blood return through the venous end are initiated, rather than by any vascular abnormality.

### Experimental Example 3: No Abnormality in Venous end Vascular Pressure

Patients C (Case 3) and D (Case 4) undergo dialysis treatment. After completion of the dialysis session, the hemodialysis device is controlled to stop withdrawing blood from the arterial end while continuing to return blood through the venous end, and at this time, the vascular pressure variations at the venous end of Patients C and D are monitored.

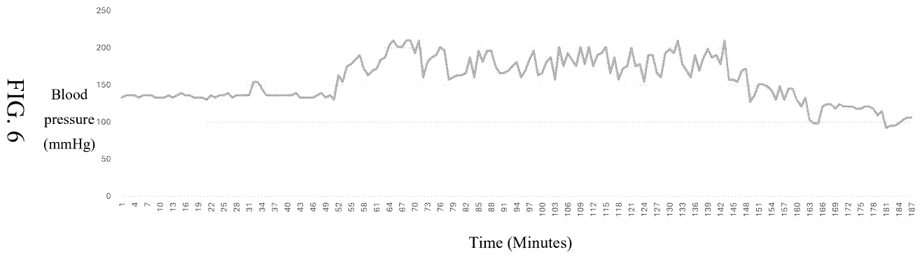

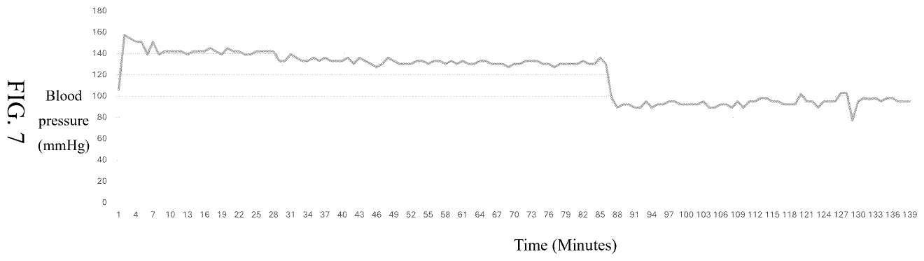

Referring to FIGS. 6 and 7, FIGS. 6 and 7 are respectively graphs showing the venous end vascular pressure variations of Patient C (Case 3) and Patient D (Case 4). As shown in FIGS. 6 and 7, for both Patient C and Patient D, during the dialysis session and after completion of the session, when the hemodialysis device is controlled to stop withdrawing blood from the arterial end while continuing to return blood through the venous end, the venous end vascular pressure remains stable with only minor fluctuations. This indicates that no vascular abnormalities are present in Patients C and D, and thus percutaneous arteriovenous fistula angioplasty is not required.

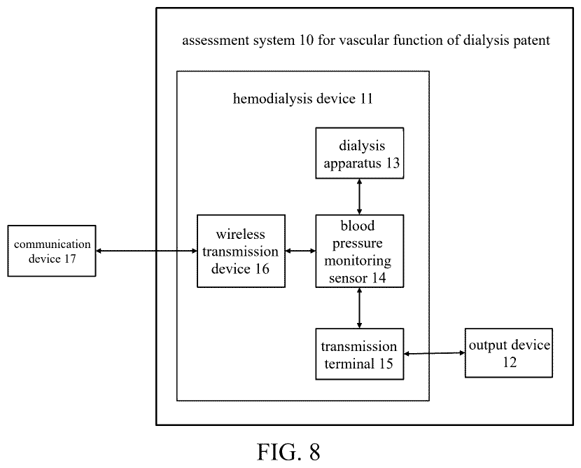

Finally, referring to FIG. 8, which is a schematic diagram of the system for evaluating vascular function in dialysis patients according to the present disclosure. As shown in FIG. 8, an assessment system 10 for vascular function of the dialysis patients includes a hemodialysis device 11 and an output device 12. The hemodialysis device 11 at least includes a dialysis apparatus 13, a blood pressure monitoring sensor 14, a transmission terminal 15, and a wireless transmission device 16. The hemodialysis device 11 is connected to the output device 12 via the transmission terminal 15, and the blood pressure monitoring sensor 14 is electrically connected to the dialysis apparatus 13, the transmission terminal 15, and the wireless transmission device 16.

The dialysis apparatus 13 is configured to perform a dialysis session on the dialysis patient, and during the dialysis session, a blood pressure of the dialysis patient is monitored using the blood pressure monitoring sensor 14, and a blood pressure monitoring status of the dialysis patient is displayed on the output device 12 via the transmission terminal 15; and after a completion of the dialysis session, the following steps are performed:

detecting a pressure change of a venous end vascular pressure of the dialysis patient within a preset time; in response to the pressure change of the venous end vascular pressure of the dialysis patient exceeding a preset pressure change range, generating a warning signal through the blood pressure monitoring sensor 14; and transmitting the warning signal to the output device 12 and/or a communication device 17.

The venous end vascular pressure of the dialysis patient in the steps performed refers to the venous end vascular pressure measured when blood is continuously returned through a venous end after a completion of a dialysis session and blood withdrawal from an arterial end is stopped.

The preset time ranges from about 5 to about 10 minutes; preferably, the preset time ranges from about 6 to about 8 minutes.

The preset pressure change range refers to an increase or decrease of at least 50% relative to an average vascular pressure value of venous end vessels of the dialysis patient; preferably, the preset pressure change range refers to an increase or decrease of at least 80% relative to an average vascular pressure value of venous end vessels of the dialysis patient.

The output device 12 may include various display interfaces, such as computer screens, monitors, or handheld device displays, but is not limited thereto.

The communication device 17 refers to a device with wired or wireless transmission capabilities, such as desktop computers, laptop computers, tablet computers, smartphones, and the like, but is not limited thereto.

When the warning signal is generated, it may be transmitted to the output device 12 via the transmission terminal 15, or alternatively, it may be transmitted to the communication device 17 via the wireless transmission device 16. For example, an application (APP) installed on the communication device may be used to receive the warning signal. This enables physicians and medical personnel to further examine the blood vessels of the dialysis patient in response to the warning signal, to determine the type of vascular abnormality present and subsequently perform appropriate treatment to address the vascular abnormality.

On the other hand, when the dialysis patient is undergoing home hemodialysis and the warning signal is generated, the communication device 17 can receive the warning signal and report it to the hospital, thereby facilitating the arrangement of an examination to determine the type of vascular abnormality present in the dialysis patient's blood vessels and subsequently perform appropriate treatment to resolve the vascular abnormality.

In summary, the assessment method for vascular function of the dialysis patient provided by the present disclosure offers significant advantages over existing methods for evaluating vascular function of dialysis patients. These advantages include simplicity and speed, high sensitivity, real-time capability, non-invasiveness, and cost-effectiveness. The assessment method of the present disclosure can effectively identify whether vascular abnormalities are present in dialysis patients, thereby enabling timely vascular intervention and improving the long-term prognosis of dialysis patients.

Those skilled in the art to which the present disclosure belongs can understand from the above contents that the present disclosure can be embodied in other specific forms without changing the technical concepts or essential characteristics of the present disclosure. In this regard, the exemplary aspects disclosed herein are for illustrative purposes only and should not be construed as limiting the scope of the present disclosure. On the contrary, the present disclosure is intended to cover not only the exemplary aspects, but also various changes, modifications, equivalents, and other aspects that may be included within the spirit and scope of the present disclosure as defined in the appended claims.

What is claimed is:

1. An assessment method for vascular function of a dialysis patient, comprising:
step S1: measuring a venous end vascular pressure of the dialysis patient by a hemodialysis device, and displaying the venous end vascular pressure on an output device;
step S2: detecting a pressure change of the venous end vascular pressure of the dialysis patient within a preset time;
step S3: in response to the pressure change of the venous end vascular pressure of the dialysis patient exceeding a preset pressure change range, generating a warning signal; and
step S4: transmitting the warning signal to the output device and/or a communication device, wherein
the venous end vascular pressure of the dialysis patient in the step S2 and the step S3 refers to the venous end vascular pressure measured when blood is continuously returned through a venous end after a completion of a dialysis session and blood withdrawal from an arterial end is stopped;
in the step S2, the venous end vascular pressure of the dialysis patient is detected by a blood pressure monitoring sensor installed in the hemodialysis device, and the preset time ranges from 5 to 10 minutes;
in the step S3, the preset pressure change range refers to an increase or decrease of at least 50% relative to an average vascular pressure value of venous end vessels of the dialysis patient, and when the venous end vascular pressure exceeds the preset pressure change range, the blood pressure monitoring sensor generates the warning signal; and
when the warning signal is generated, blood vessels of the dialysis patient are evaluated for a presence of at least one abnormality.

2. The assessment method of claim 1, wherein the at least one abnormality is selected from vascular stenosis, vascular occlusion and vascular sclerosis.

3. An assessment system for vascular function of a dialysis patient, the assessment system comprising:
a hemodialysis device at least comprising a dialysis apparatus, a blood pressure monitoring sensor, a transmission terminal, and a wireless transmission device; and
an output device connected to the hemodialysis device via the transmission terminal, wherein
the blood pressure monitoring sensor is electrically connected to the dialysis apparatus, the transmission terminal, and the wireless transmission device.

4. The assessment system of claim 3, wherein the dialysis apparatus is configured to perform a dialysis session on the dialysis patient, and during the dialysis session, a blood pressure of the dialysis patient is monitored using the blood pressure monitoring sensor, and a blood pressure monitoring status of the dialysis patient is displayed on the output device via the transmission terminal; and after a completion of the dialysis session, the following steps are performed:
detecting a pressure change of a venous end vascular pressure of the dialysis patient within a preset time;
in response to the pressure change of the venous end vascular pressure of the dialysis patient exceeding a preset pressure change range, generating a warning signal through the blood pressure monitoring sensor; and
transmitting the warning signal to the output device and/or a communication device.

5. The assessment system of claim 4, wherein the venous end vascular pressure of the dialysis patient refers to the venous end vascular pressure measured when blood is continuously returned through a venous end after a completion of a dialysis session and blood withdrawal from an arterial end is stopped.

6. The assessment system of claim 5, wherein the preset time ranges from 5 to 10 minutes.

7. The assessment system of claim 5, wherein the preset pressure change range refers to an increase or decrease of at least 50% relative to an average vascular pressure value of venous end vessels of the dialysis patient.

8. The assessment system of claim 4, wherein, when the warning signal is generated, blood vessels of the dialysis patient are evaluated for a presence of at least one abnormality.

9. The assessment system of claim 8, wherein the at least one abnormality is selected from vascular stenosis, vascular occlusion and vascular sclerosis.
